(19) United States
(12) Patent Application Publication
(10) Pub. No.: US 2010/0319584 A1
(43) Pub. Date: Dec. 23, 2010

(54) MODIFICATION ADDITIVE FOR ASPHALT MASSES AND TYPE AND MANNER OF ASPHALT PRODUCTION FOR THE CREATION OF MINERAL-ASPHALT MIXTURES HAVING A HIGH SHEAR MODULUS

(76) Inventors: Wlodzimierz Górniak, Czestochowa (PL); Piotr Górniak, Czestochowa (PL); Dariusz Kowal, Czestochowa (PL); Antoni Wójcik, Czestochowa (PL); Hubert Zymek, Koniecpol (PL)

Correspondence Address: Horst M Kasper, 13 Forest Drive, Warren, NJ, 07059, US

(21) Appl. No.: 12/736,032

(22) PCT Filed: Feb. 26, 2009
(86) PCT No.: PCT/PL2009/000017

§ 371 Date: Sep. 1, 2010

(30) Foreign Application Priority Data
Mar. 3, 2008 (PL) P - 384 595

(51) Int. Cl. C04B 24/36; C07C 13/15

(52) U.S. Cl. 106/671; 585/17

(57) ABSTRACT

The invention relates to a modification additive for asphalt masses and to the manner of production of asphalt masses for the creation of mineral-asphalt mixtures having a high shear modulus. The essence of the invention is a modifier, characterized in that it comprises 50 to 97% of cyclopentadiene oligomers, which are dissolved in 2 to 30% by weight of alkenes with 9-11 carbon atoms in a molecule, and 1 to 15% by weight of cyclic and aromatic olefins, wherein the latter are segregation residues of other hydrocarbon fractions. The liquid asphalt mass is added to the above-mentioned modifier in a quantity of 0.1 to 15% by weight at a temperature of no more than 185° C. and kept in a liquid state for at least 15 minutes. The modified asphalt obtained in this way, together with the mineral additive, forms mineral-asphalt mixtures having a high shear modulus.

The subject of the invention is a modifying agent for asphalts and manner of producing asphalts for asphalt-concrete mixes with high shear rigidity. Patent no. 363816 describes a modifying agent for producing asphalt-concrete mixes comprising by weight 50-70% unsaturated C₅ hydrocarbons, 30-45% stabilizer in the form of benzene solution of cyclic non-aromatic C₄ and C₈ hydrocarbons in the amount of up to 20% by weight with respect to benzene and 0-10% ethanol. Another Polish patent no. 317685 outlines a liquid, thermally stable modifying agent for hot mix road asphalts comprising by weight 0.5-20% of high-boiling point oils with solidification point of below 10° C., 0.05-5% of N,N-di-(polyoxyethylene)-alkylamines comprising 12-22 carbon atoms in the alkyl chain and 4-15 oxyethylene constituents in the molecule, 0.025-5% of thermoplastic, styrene-butadiene-styrene elastomer and up to 100% of alkylaminepolyamines and/or 2-alkyl imidazoline with alkyl chain comprising 12-22 carbon atoms. The method is known for producing an elastomer-asphalt binding agent under patent no. 336806, comprising, among others, the styrene-butadiene-styrene and/or styrene-isoprene-styrene in the amount of 1-8%. A modified petroleum asphalt is known under patent 373932 which, by weight, consists of 0,5-30% post-pyrolytic oil fractions being a by-product of olefin pyrolysis of gasoline characterised by boiling point of above 300° C. and plasticising temperature of above 50° C.

Application of known modifying agents for asphalts mixes as well as manners of production of modified asphalts, considering generally high cost of their production and durability characteristics expressed by shear rigidity, is unfavourable. Besides, high-temperature processing and laying of such mixes results in environmental release of toxic and carcinogenic benzene and benzene derivatives.

The purpose of the invention was to design such modifying agent and manner of producing asphalt mix that the end-product in the form of asphalt-concrete mix was characterised by very good mechanical properties, was environmentally safe and showed no negative influence on the persons directly involved in production and forming of asphalt-concrete mixes.

The nature of the invention is the modifying agent in the form of a mixture of organic compounds containing, by weight, 50-97% of cyclopentadiene oligomers, 2-30% of C₉-C₁₁ alkenes and 1-15% of cyclic aromatic olefins being a residual product of fractional separation of other hydrocarbon fractions. In order to obtain asphalts that are combined with mineral aggregate to form asphalt-concrete mixes with high shear rigidity as set out in ZS-WMS 2007 (IBDiM), a modifying agent with the composition as described above should be mixed with the asphalt binding agent in the following manner:
1. To a tank with liquid asphalt the modifying agent is added in the amount of 0.1-15% by weight, while constant stirring and a temperature below 185° C.
2. The modifying agent is added in the amount of 0.1-15% by weight, successively during pumping of asphalt between two tanks and/or to a tanker, whereby during the pumping intensive mixing takes place and under maximum temperature of 185° C.

In both cases, the mixture should be in the liquid state for at least 15 minutes.

### EXAMPLE 1

To a tank containing liquid melted asphalt the following ingredients are added in the following amounts by weight: 0.2% modifying agent composed of 70% cyclopentadiene oligomers, 25% of C₉-C₁₁ alkenes and 5% of cyclic aromatic olefins being a residual product of fractional separation of other hydrocarbon fractions, after which the mixing should be continued for 2 hours under the temperature of 140° C. An asphalt thus mixed complies with the parameters set out in the norm PN-EN-12591:2002 for class D 35/50 asphalt.

### EXAMPLE 2

To a tank containing liquid melted asphalt the following ingredients are added in the following amounts by weight: 10% modifying agent composed of 90% cyclopentadiene oligomers, 7% of C₉-C₁₁ alkenes and 3% of cyclic aromatic olefins being a residual product of fractional separation of other hydrocarbon fractions, after which the mixing should be continued for 30 minutes under the temperature of 170° C. An asphalt thus mixed complies with the parameters set out in the norm PN-EN-12591:2002 for class D 250/330 asphalt.

1. A modifying agent for asphalts characterised in that it contains, by weight, 50-97% cyclopentadiene oligomers solved in 2-30% of C₉-C₁₁ alkenes and 1-15% of cyclic aromatic olefins being a residual product of fractional separation of other hydrocarbon fractions.

2. The manner of producing an asphalt binding agent which is combined with mineral aggregate to form asphalt-concrete mixes with high shear rigidity characterised in that a modifying agent is added to liquid asphalt, modifying agent being premixed as set out in claim 1 and being added in concentrations between 0.1% and 15% by weight, while maintaining constant mixing in the maximum temperature of 185° C., whereby the mix should be kept in the liquid state for at least 15 minutes.
